(19) United States
(12) Patent Application Publication
(10) Pub. No.: US 2010/0260674 A1
(43) Pub. Date: Oct. 14, 2010

(54) QUINAZOLINE DERIVATIVES AND METHODS OF TREATMENT

(75) Inventor: Roger Tung, Lexington, MA (US)

Correspondence Address: EDWARDS ANGELL PALMER & DODGE LLP, P.O. BOX 55874, BOSTON, MA, 02205, US

(73) Assignee: Concert Pharmaceuticals, Inc., Lexington, MA (US)

(21) Appl. No.: 12/694,249

(22) Filed: Jan. 26, 2010

Related U.S. Application Data
(63) Continuation-in-part of application No. 11/957,442, filed on Dec. 15, 2007.
(60) Provisional application No. 60/875,320, filed on Dec. 15, 2006.
(60) Provisional application No. 61/147,458, filed on Jan. 26, 2009.

(51) Int. Cl. A61K 31/517; C07D 239/94; A61K 39/395; A61K 51/00; A61K 38/21; A61K 31/192; A61K 31/69; A61K 31/593; A61K 31/5377; A61K 31/7068; A61K 31/282; A61K 31/415; A61K 31/24; A61K 33/24; A61K 31/496; A61K 31/7048; A61K 31/4545; A61K 31/436; A61K 31/56; A61K 31/352; A61K 31/4375; A61K 31/454; A61K 31/4196; A61K 31/4985; A61K 31/337; A61K 38/22; A61K 31/519; A61K 31/4412; A61K 31/05; A61K 31/404; A61K 31/33; A61K 31/4709; A61K 31/19; A61K 31/437; A61K 31/16; A61P 17/06; A61P 35/00; A61P 37/00; A61P 13/12; A61P 9/00

(52) U.S. Cl. 424/1.89; 544/293; 514/266.4; 424/133.1; 424/85.7; 514/569; 514/64; 514/167; 514/234.5; 514/49; 514/492; 514/406; 514/542; 424/649; 514/252.19; 514/26; 514/318; 514/27; 514/291; 514/171; 514/456; 514/252.18; 514/283; 514/266.24; 514/323; 514/383; 514/249; 514/449; 424/142.1; 424/85.1; 514/265.1; 514/350; 514/731; 514/414; 514/183; 514/312; 514/557; 514/281; 514/616

(57) ABSTRACT

This invention relates to novel quinazoline derivatives, and their pharmaceutically acceptable salts. The invention also provides compositions comprising a compound of this invention and the use of such compositions in methods of treating diseases and conditions beneficially treated by inhibiting cell surface tyrosine receptor kinases.

### RELATED APPLICATIONS

This application is a continuation-in-part of U.S. patent application Ser. No. 11/957,442, filed Dec. 15, 2007, pending, which claims the benefit of U.S. Provisional Patent Application No. 60/875,320, filed Dec. 15, 2006. This application also claims the benefit of U.S. Provisional Patent Application No. 61/147,458, filed Jan. 26, 2009. The contents of each of these applications are incorporated herein by reference.

### TECHNICAL FIELD OF THE INVENTION

This invention relates to novel quinazoline derivatives, and their pharmaceutically acceptable salts. The invention also provides compositions comprising a compound of this invention and the use of such compositions in methods of treating diseases and conditions beneficially treated by inhibiting cell surface tyrosine receptor kinases.

Quinazoline derivatives which bear at the 4-position an anilino substituent and which also bear an alkoxy substituent at the 7-position and an alkoxy substituent at the 6-position, are disclosed inter alia in U.S. Pat. No. 5,747,498, EP 1,110,953, EP 817,775, and U.S. Pat. No. 6,476,040. One of those derivatives, erlotinib, is known chemically as [6,7-Bis-(2-methoxy-ethoxy)-quinazolin-4-yl]-(3-ethynyl-phenyl)-amine and as N-(3-ethynylphenyl)-6,7-bis(2-methoxyethoxy)quinazolin-4-amine.

Erlotinib is an inhibitor of tyrosine kinases, particularly EGF receptor tyrosine kinases. Erlotinib has been approved in the United States in and in Europe for the treatment of locally advanced or metastatic non-small cell lung cancer (NSCLC) after failure of at least one prior chemotherapy regimen. Erlotinib is also approved in the United States in combination with gemcitabine, for the treatment of metastatic pancreatic cancer. Clinical trials are ongoing investigating the use of erlotinib alone or in combination with other agents for the treatment of a variety of cancers, including non-small cell lung cancer, ovarian cancer, colorectal cancer, head and neck cancer, brain cancer, bladder cancer, sarcoma, prostate cancer, melanoma, cervical cancer, solid tumors, astrocytoma, breast cancer, pancreatic cancer, glioblastoma multiform, renal cancer, digestive/gastrointestinal cancer, liver cancer, and gastric cancer. Erlotinib is also thought to be useful in the treatment of benign hyperplasia of the skin (psoriasis) or prostate (BPH).

Despite the beneficial activities of erlotinib, there is a continuing need for new compounds to treat the aforementioned diseases and conditions.

### DEFINITIONS

The terms “ameliorate” and “treat” are used interchangeably and both mean decrease, suppress, attenuate, diminish, arrest, or stabilize the development or progression of a disease (e.g., a disease or disorder delineated herein).

By “disease” is meant any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of erlotinib will inherently contain small amounts of deuterated isotopologues. The concentration of naturally abundant stable hydrogen isotopes, notwithstanding this variation, is small and immaterial with respect to the degree of stable isotopic substitution of compounds of this invention. See, for instance, Wada, E et al., Seikagaku, 1994, 66: 15; Ganes, L Z et al., Comp Biochem Physiol Mol Integr Physiol, 1998, 119: 725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

The term “isotopologue” refers to species that differ from a specific compound of this invention only in the isotopic composition of their molecules or ions.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 55% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

A salt of a compound of this invention is formed between an acid and a basic group of the compound, such as an amino functional group, or a base and an acidic group of the compound, such as a carboxyl functional group. According to another preferred embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

The compounds of the present invention may contain one or more asymmetric carbon atoms. As such, a compound of this invention can exist as the individual stereoisomers (enantiomers or diastereomers) as well a mixture of stereoisomers. Accordingly, a compound of the present invention will include not only a stereoisomeric mixture, but also individual respective stereoisomers substantially free from one another stereoisomers. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present Methods of obtaining or synthesizing diastereomers are well known in the art and may be applied as practicable to final compounds or to starting material or intermediates. Other embodiments are those wherein the compound is an isolated compound. The term “at least X % enantiomerically enriched” as used herein means that at least X % of the compound is a single enantiomeric form, wherein X is a number between 0 and 100, inclusive.

The term “stable compounds”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

The terms “lighter isotopologue” and “lighter atom isotopologue” as used herein, refer to species that differ from a specific compound of this invention in that they comprise hydrogen at positions occupied by a deuterium in the specific compound.

“D” and “d” both refer to deuterium.

“Stereoisomer” refers to both enantiomers and diastereomers.

“Tert”, “^{t}”, and “t-” each refer to tertiary.

“US” refers to the United States of America.

“FDA” refers to Food and Drug Administration.

“NDA” refers to New Drug Application.

Throughout this specification, reference to “each Y” includes, independently, all “Y” groups (e.g., Y^{1a}, Y^{1b}, Y^{1c}, Y^{2a}, Y^{2b}, and Y^{2b}) where applicable. Throughout this specification, reference to “each Z” includes, independently, all “Z” groups (e.g., Z^{1a}, Z^{1b}, Z^{2a}, and Z^{2b}) where applicable.

### Therapeutic Compounds

In one embodiment, the present invention provides a compound of Formula I:

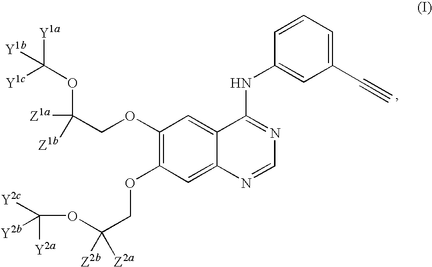

or a pharmaceutically acceptable salt of said compound, wherein:

each Y (e.g., Y^{1a}, Y^{1b}, Y^{1c}, Y^{2a}, Y^{2b}, and Y^{2c}) is independently selected from hydrogen or deuterium;

each Z (e.g., Z^{1a}, Z^{1b}, Z^{2a}, and Z^{2b}) is independently selected from hydrogen or deuterium;

and at least one Y or Z is deuterium.

In one embodiment, the invention provides a compound of Formula I, wherein Y^{1a}, Y^{1b}, and Y^{1c} are simultaneously deuterium. In another embodiment, the invention provides a compound of Formula I, wherein Y^{2a}, Y^{2b} and Y^{2c} are simultaneously deuterium. In still another embodiment, the invention provides a compound of Formula I, wherein Y^{1a}, Y^{1b}, Y^{1c}, Y^{2a}, Y^{2b} and Y^{2c} are simultaneously deuterium.

In another embodiment, the invention provides a compound of Formula I, wherein each Z¹ is the same; and each Z² is the same.

In another embodiment, the invention provides a compound of Formula I, wherein Z^{1a} and Z^{1b} are simultaneously deuterium.

In another embodiment, the invention provides a compound of Formula I, wherein Z^{2a} and Z^{2b} are simultaneously deuterium.

In another embodiment, the invention provides a compound of Formula I, wherein Z^{1a}, Z^{1b}, Z^{2a} and Z^{2b} are simultaneously deuterium.

In another embodiment, the invention provides a compound of Formula I, wherein each Y¹ and each Z¹ is simultaneously deuterium.

In another embodiment, the invention provides a compound of Formula I, wherein each Y² and each Z² is simultaneously deuterium.

Specific compounds of Formula I include those delineated in Table 1 below.

TABLE 1

| Cmpd | Y^{1a} | Y^{1b} | Y^{1c} | Y^{2a} | Y^{2b} | Y^{2c} | Z^{1a} | Z^{1b} | Z^{2a} | Z^{2b} |
|---|---|---|---|---|---|---|---|---|---|---|
| 97 | H | H | H | D | D | D | D | D | D | D |
| 98 | D | D | D | H | H | H | D | D | D | D |
| 99 | D | D | D | D | D | D | D | D | H | H |
| 100 | D | D | D | D | D | D | H | H | D | D |
| 101 | D | D | D | H | H | H | H | H | H | H |
| 102 | H | H | H | D | D | D | H | H | H | H |
| 103 | H | H | H | H | H | H | D | D | H | H |
| 104 | H | H | H | H | H | H | H | H | D | D |
| 105 | D | D | D | D | D | D | H | H | H | H |
| 106 | H | H | H | H | H | H | D | D | D | D |
| 107 | D | D | D | H | H | H | D | D | H | H |
| 108 | H | H | H | D | D | D | H | H | D | D |
| 109 | D | D | D | D | D | D | D | D | D | D |

In another embodiment, the invention provides a compound of Formula IA:

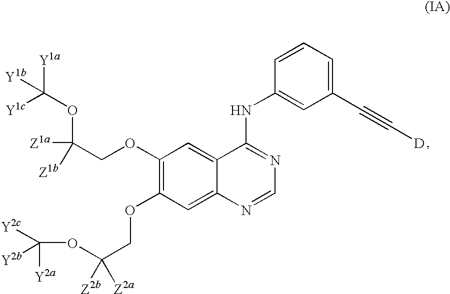

wherein each Y (e.g., Y^{1a}, Y^{1b}, Y^{1c}, Y^{2a}, Y^{2b}, and Y^{2c}) and each Z (e.g., Z^{1a}, Z^{1b}, Z^{2a}, and Z^{2b}) is independently selected from hydrogen and deuterium.

In one embodiment, the invention provides a compound of Formula IA, wherein each Y¹ is the same; and each Y² is the same. In one specific embodiment, Y^{1a}, Y^{1b}, and Y^{1c} are simultaneously deuterium. In another specific embodiment, Y^{2b} and Y^{2c} are simultaneously deuterium. In still another specific embodiment, Y^{1a}, Y^{1b}, Y^{1c}, Y^{2a}, Y^{2b} and Y^{2c} are simultaneously deuterium.

In another embodiment, the invention provides a compound of Formula IA, wherein each Z¹ is the same; and each Z² is the same. In a specific embodiment Z^{1a} and Z^{1b} are simultaneously deuterium. In another specific embodiment, Z^{2a} and Z^{2b} are simultaneously deuterium. In still another specific embodiment, Z^{1a}, Z^{1b}, Z^{2a} and Z^{2b} are simultaneously deuterium.

In another embodiment, the invention provides a compound of Formula IA, wherein each Y¹ and each Z¹ is simultaneously deuterium.

In another embodiment, the invention provides a compound of Formula IA, wherein each Y² and each Z² is simultaneously deuterium.

In still another specific embodiment of Formula IA, each Y and each Z is simultaneously hydrogen.

Specific compounds of Formula IA include those delineated in Table 1 below.

TABLE 2

| Cmpd | Y^{1a} | Y^{1b} | Y^{1c} | Y^{2a} | Y^{2b} | Y^{2c} | Z^{1a} | Z^{1b} | Z^{2a} | Z^{2b} |
|---|---|---|---|---|---|---|---|---|---|---|
| 110 | H | H | H | H | H | H | H | H | H | H |
| 111 | D | D | D | H | H | H | H | H | H | H |
| 112 | H | H | H | D | D | D | H | H | H | H |
| 113 | H | H | H | H | H | H | D | D | H | H |
| 114 | H | H | H | H | H | H | H | H | D | D |
| 115 | D | D | D | D | D | D | H | H | H | H |
| 116 | H | H | H | H | H | H | D | D | D | D |
| 117 | D | D | D | H | H | H | D | D | H | H |
| 118 | H | H | H | D | D | D | H | H | D | D |
| 119 | D | D | D | D | D | D | D | D | D | D |
| 120 | H | H | H | D | D | D | D | D | D | D |
| 121 | D | D | D | H | H | H | D | D | D | D |
| 122 | D | D | D | D | D | D | D | D | H | H |
| 123 | D | D | D | D | D | D | H | H | D | D |

In another set of embodiments, any atom not designated as deuterium in any of the embodiments set forth above is present at its natural isotopic abundance.

In an even more specific embodiment, the compound of this invention is selected from:

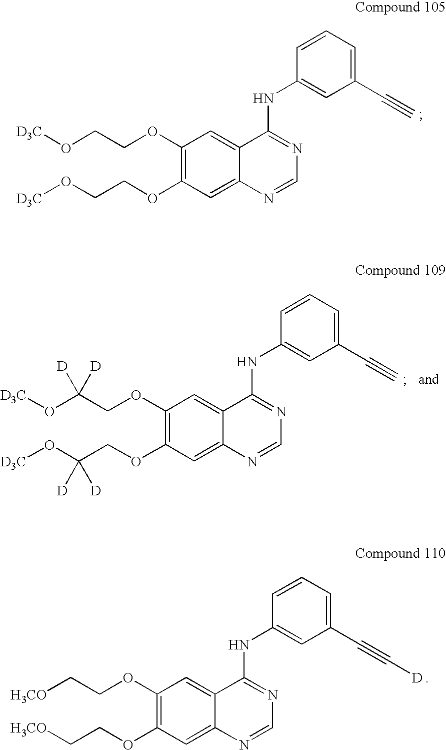

The synthesis of compounds of the formulae herein (e.g., Formula I and IA) can be readily effected by synthetic chemists of ordinary skill Relevant procedures and intermediates are disclosed, for instance, in U.S. Pat. No. 5,747,498, EP 1,110,953, EP 817,775, and U.S. Pat. No. 6,476,040. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

Convenient methods for producing compounds of Formula I are described in Schemes 1-7. In each of the schemes set forth below, asterisks (*) are used to designate optional deuteration sites, each “P” is used to designate an independently selected protecting group (e.g., nitrogen-protecting group, tBOC, benzyl, acyl), and each “L” is used to designate a displaceable group. A suitable displaceable group L is, for example, a halogen, alkoxy, aryloxy or sulfonyloxy group, for example a chloro, bromo, methoxy, phenoxy, methanesulfonyloxy or toluene-4-sulfonyloxy group. Each R is an H, alkyl, alkoxyalkyl or protecting group.

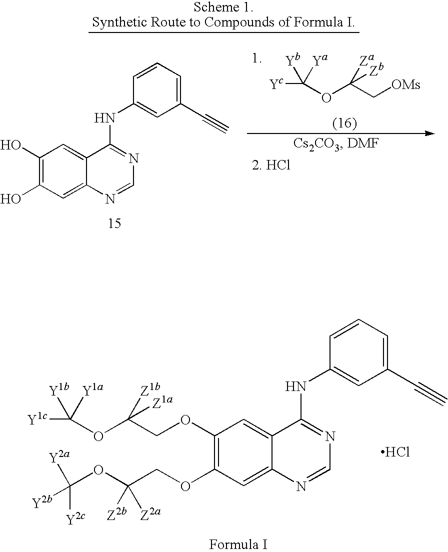

Scheme 1 outlines a route to compounds of Formula I, wherein the quinazolinamine 15 (prepared as described in Ramanadhan, J P et al., WO 2007060691A2) may be combined with an appropriately deuterated 2-methoxyethyl methane sulfonate 16 to form a compound of Formula I.

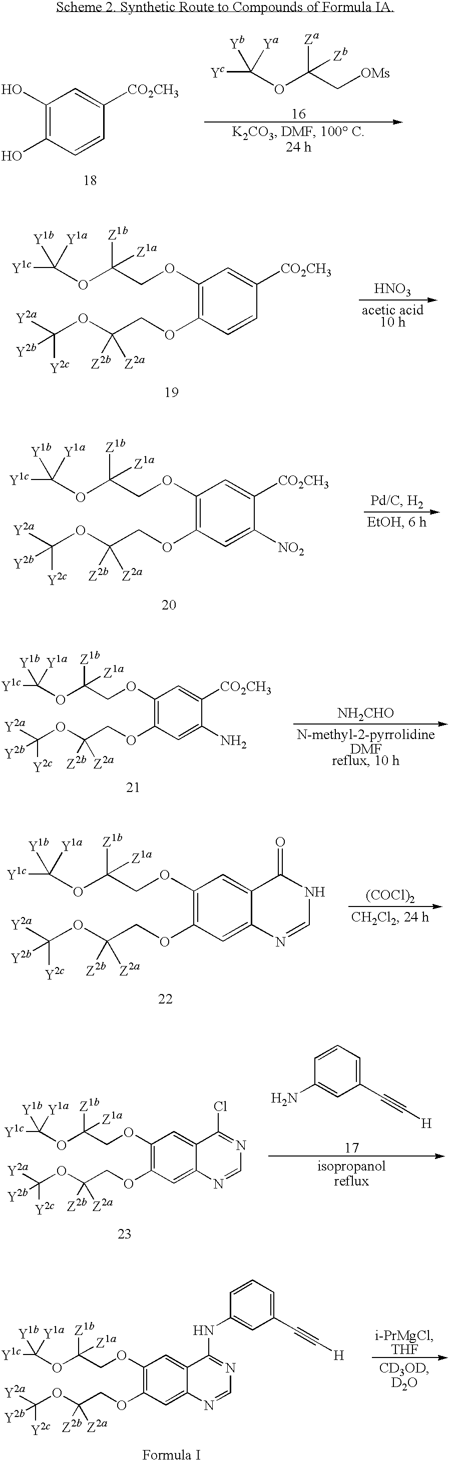

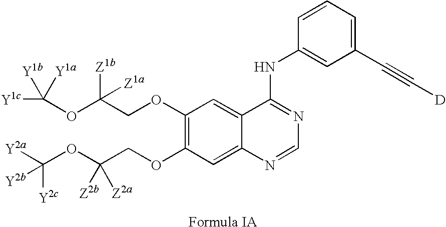

Scheme 2 outlines a synthetic route to compounds of Formula IA based on that described by Knesl, P et al. in Molecules, 2006, 11: 286-297. Methyl-3,4-dihydroxy benzoate 18 may be combined with an appropriately deuterated 2-methoxyethyl methane sulfonate 16 to produce intermediate 19. Nitration of 19 may be carried out with nitric acid and acetic acid to produce the nitrobenzene compound 20. The nitrobenzene 19 may be reduced to the corresponding aniline 21, which then may be cyclized to quinazolinone 22. The quinazolinone 22 may be chlorinated with oxalyl chloride to form the chloroquinazoline 23, which then may be combined with ethynyl aniline 17 to form a compound of Formula I. Conversion to a compound of Formula IA may be achieved by treatment with isopropyl magnesium chloride in the presence of d₃-MeOH and D₂O. This conversion step may be used on a compound of Formula I produced as outlined in Schemes 1.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R, R′, X, etc.) or not. The suitability of a chemical group in a compound structure for use in synthesis of another compound structure is within the knowledge of one of ordinary skill in the art.

Additional methods of synthesizing compounds of the formulae herein and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides compositions comprising an effective amount of a compound of the formulae herein (e.g., Formula I), or a pharmaceutically acceptable salt, of said compound; and an acceptable carrier. In one embodiment, the composition is a pyrogen-free composition. In another embodiment, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

A specialized formulation for compounds of the formulae herein (e.g., Formula I or IA) is a nanoparticulate formulation as disclosed for example in WO 2006110811.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardley, N.Y., 1970, 537. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

The size of the dose required for the therapeutic or prophylactic treatment of a particular proliferative disease will necessarily be varied depending on the patient treated, the route of administration, and the severity of the illness being treated. Such dosages can be found in U.S. Pat. No. 5,770,599. The compounds of the invention will normally be administered to a patient at a unit dose within the range of about 5 mg to about 10,000 mg per square meter body area of the patient, i.e. from about 0.1 mg/kg to about 200 mg/kg, providing a therapeutically-effective dose. A unit dose form such as a tablet or capsule will usually contain, for example from about 1 mg to about 250 mg of active ingredient. Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of the present invention further comprises a second therapeutic agent. The second therapeutic agent includes any compound or therapeutic agent known to have or that demonstrates advantageous properties when administered with erlotinib. Such agents are described in detail in U.S. Pat. No. 5,770,599; WO 2001/076586; WO 2002/005791; WO 2001/070255; WO 2003/088971; WO 2004/014426; WO 2005/000213; WO 2005/004872; WO 2005/046665; WO 2005/052005; WO 2005/117888; WO 2006/026313; WO 2004/035057; and WO 2006/099396; the disclosures of which are incorporated herein by reference.

In one embodiment, the second therapeutic agent is selected from 2-deoxy-2-[18F]fluoro-D-glucose, 3′-deoxy-3′-[18F]fluorothymidine, 5-fluorouracil, AV412, avastin, bevacizumab, bexarotene, bortezomib, calcitriol, canertinib, capecitabine, carboplatin, celecoxib, cetuximab, CHR-2797, cisplatin, dasatinib, digoxin, enzastaurin, etoposide, everolimus, fulvestrant, gefitinib, gemcitabine, genistein, imatinib, irinotecan, lapatinib, lenalidomide, letrozole, leucovorin, matuzumab, oxaliplatin, paclitaxel, panitumumab, pegfilgrastim, pegylated alfa-interferon, pemetrexed, Polyphenon® E, satraplatin, sirolimus, sorafenib, sutent, sulindac, sunitinib, taxotere, temodar, temozolomide, temsirolimus, TG01, tipifarnib, trastuzumab, valproic acid, vinflunine, volociximab, vorinostat, and XL647.

In a more specific embodiment, the second therapeutic agent is bevacizumab.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from a daily dose in the range of from about 1 mg/kg to about 100 mg/kg is employed. In certain embodiments, a compound of the invention or a pharmaceutically-acceptable salt thereof, will be administered at a daily dose of about 1 mg/kg to about 20 mg/kg; preferably about 1 mg/kg to about 5 mg/kg is employed. In certain embodiments, a unit dose in the range of about 1 mg/kg to about 200 mg/kg, preferably about 1 mg/kg to about 100 mg/kg, more preferably about 1 mg/kg to about 10 mg/kg, is envisaged.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for erlotinib.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

If the second therapeutic agents referenced above act synergistically with the compounds of this invention it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

According to another embodiment, the invention provides a method of treating a patient suffering from or susceptible to a disease that is beneficially treated by erlotinib comprising the step of administering to the patient in need thereof an effective amount of a compound or a composition of this invention. Such diseases are well known in the art and are disclosed, for example, in U.S. Pat. No. 5,770,599, U.S. Pat. No. 5,747,498, EP 1,110,953, EP 817,775, and U.S. Pat. No. 6,476,040. In particular, the invention provides a method of treating a patient suffering from or susceptible to cancer, inflammation, angiogenesis, vascular restenosis, immunological disorder, pancreatitis, kidney disease, blastocyte maturation and implantation, psoriasis, or benign prostatic hypertrophy (BPH).

In a more specific embodiment, the cancer is selected from non-small cell lung cancer, ovarian cancer, colorectal cancer, head and neck cancer, brain cancer, bladder cancer, sarcoma, prostate cancer, melanoma, cervical cancer, solid tumors, astrocytoma, breast cancer, pancreatic cancer, glioblastoma multiform, renal cancer, digestive/gastrointestinal cancer, liver cancer, and gastric cancer.

In an even more specific embodiment, the cancer is non-small cell lung cancer.

The compounds of the invention also have utility in the treatment of additional disorders of cellular growth in which aberrant cell signaling by way of receptor tyrosine kinase enzymes or non-receptor tyrosine kinase enzymes, including as yet unidentified tyrosine kinase enzymes, are involved. Such disorders include, for example, inflammation, angiogenesis, vascular restenosis, immunological disorder, pancreatitis, kidney disease and blastocyte maturation and implantation. Additionally, the compounds of the invention can be used to treat other diseases involving excessive cellular proliferation such as psoriasis and benign prostatic hypertrophy (BPH).

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with erlotinib. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include a method of treating a patient suffering from or susceptible to cancer comprising the step of co-administering a compound of Formula I and a second therapeutic agent selected from 2-deoxy-2-[18F]fluoro-D-glucose, 3′-deoxy-3′-[18F]fluorothymidine, 5-fluorouracil, AV412, avastin, bevacizumab, bexarotene, bortezomib, calcitriol, canertinib, capecitabine, carboplatin, celecoxib, cetuximab, CHR-2797, cisplatin, dasatinib, digoxin, enzastaurin, etoposide, everolimus, fulvestrant, gefitinib, gemcitabine, genistein, imatinib, irinotecan, lapatinib, lenalidomide, letrozole, leucovorin, matuzumab, oxaliplatin, paclitaxel, panitumumab, pegfilgrastim, pegylated alfa-interferon, pemetrexed, Polyphenon® E, satraplatin, sirolimus, sorafenib, sutent, sulindac, sunitinib, taxotere, temodar, temozolomide, temsirolimus, TG01, tipifarnib, trastuzumab, valproic acid, vinflunine, volociximab, vorinostat, and XL647 to the patient in need thereof.

In a more specific embodiment, the co-administered second therapeutic agent is bevacizumab.

In an even more specific embodiment, the co-administered second therapeutic agent is bevacizumab and the patient is suffering from non-small cell lung cancer.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a patient, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In another aspect, the invention provides the use of a compound of the formulae herein, or a pharmaceutically-acceptable salt thereof, in the manufacture of a medicament for use in the production of an anti-proliferative effect in a patient.

In another embodiment, the invention provides a method of modulating the activity of cell surface tyrosine receptor kinases, including epidermal growth factor receptor kinases (EGFR), in a cell comprising contacting the cell with one or more compounds of any of the formulae herein.

In yet another aspect, the invention provides the use of a compound of the formulae herein (e.g., Formula I or IA) alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of the formulae herein for use in the treatment or prevention in a patient of a disease, disorder or symptom thereof delineated herein.

### Diagnostic Methods and Kits

The present invention also provides kits for use to treat non-small cell lung cancer, ovarian cancer, colorectal cancer, head and neck cancer, brain cancer, bladder cancer, sarcoma, prostate cancer, melanoma, cervical cancer, solid tumors, astrocytoma, breast cancer, pancreatic cancer, glioblastoma multiform, renal cancer, digestive/gastrointestinal cancer, liver cancer, or gastric cancer. These kits comprise (a) a pharmaceutical composition comprising a compound of Formula I or IA or a pharmaceutically acceptable salt thereof, wherein said pharmaceutical composition is in a container; and (b) instructions describing a method of using the pharmaceutical composition to treat the cancer.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### EXAMPLES

### Example 1

### Synthesis of Deuterated Methoxyethyl Methanesulfonate 16

Reagent 16 was prepared as outlined in Scheme 3A or 3B.

### A. Synthesis of (Methoxy-d₃)ethyl Methanesulfonate via Scheme 3A

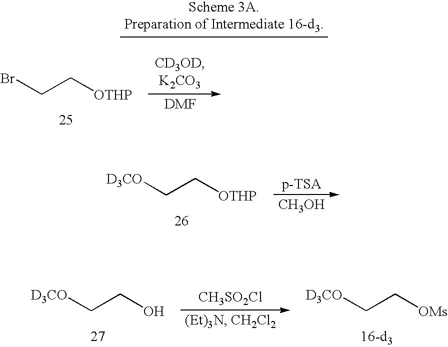

Synthesis of 2-(2-(Methoxy-d₃)ethoxy)tetrahydro-2H-pyran (26). To a solution of 2-(2-bromoethoxy)tetrahydro-2H-pyran, 25 (40.0 g, 24.3 mmol, commercially available) in DMF (100 mL) was added K₂CO₃ (40.24 g, 48.0 mmol) and CD₃OD (4.23 g, 29.2 mmol) and the resulting mixture was stirred at 60° C. for 12.0 h. The reaction mixture was poured into cold water and extracted with diethyl ether (2×150 mL). The combined organic extracts were washed with brine, dried over Na₂SO₄ and concentrated under reduced pressure to give the product 26 (11.50 g, 37%).

Synthesis of 2-(Methoxy-d₃)ethanol (27). To a solution of 26 (11.0 g, 6.77 mmol) in methanol (25 mL) was added p-TSA.H₂O (100 mg). The resulting mixture was stirred at room temperature (rt) for 10.0 h. Methanol was removed via distillation at atmospheric pressure and the residue was distilled at 140° C. to give the 2-(methoxy-d₃) ethanol, 27 (3.00 g, 57%).

Synthesis of 2-(Methoxy-d₃)ethyl Methanesulfonate (16-d₃). To a solution of 2-(methoxy-d₃)ethanol, 27 (2.80 g, 36.8 mmol) and triethyl amine (4.80 mL) in methylene chloride (50 mL) cooled to 0° C., was added methane sulfonylchloride (4.10 g, 37 mmol). The resulting mixture was stirred at 0° C. for 2.0 h. The mixture was then washed with aq NaHCO₃ and brine, dried over Na₂SO₄ and concentrated under reduced pressure to give the product 16-d₃ (3.05 g, 55%). ¹H NMR (CDCl₃): δ 2.94 (s, 3H), 3.66 (m, 2H), 3.80 (t, 2H).

### B. Synthesis of Deuterated Methoxy Methanesulfonates Via Scheme 3B

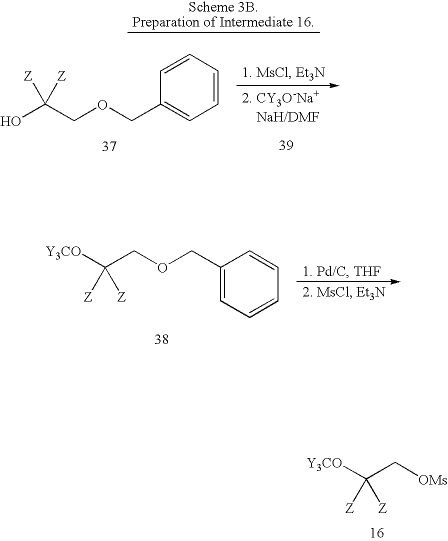

I. 2-(Methoxy-d₃)-2,2-d₂-ethyl Methanesulfonate (16-d₅). Intermediate (16-d₅ was synthesized as outlined in Scheme 3B, above.

Synthesis of (2-(Methoxy-d₃)-2,2-d₂-ethoxy)methyl)benzene (38-d₅). To a solution of 2-benzyloxy-1,1-d₂-ethanol 37-d₂ (5.00 g, 32.2 mmol, synthesized according to the procedure described by Bird, I et al in J Label Comp Radiopharm, 1989, 27(2): 199-216) in methylene chloride (50.0 mL) was added methanesulfonyl chloride (4.00 mL, 48.3 mmol) and triethylamine (6.70 mL, 48.3 mmol) and the solution was stirred at room temperature (“rt”) for 1 h. Water (25 mL) was added to the reaction mixture and the organic layer was separated, washed with satd. brine solution (15 mL), dried over Na₂SO₄ and evaporated. The resulting crude mesylate intermediate (6.00 g, 25.5 mmol, 80%) was dissolved in DMF (20.0 mL) and combined with CD₃O⁻Na⁺39, which was produced in situ by incubating CD₃OD [isotopic purity 99.8%, Aldrich] (2.00 mL, 51.0 mmol) in DMF (30.0 mL) with sodium hydride (2.00 g, 84.19 mmol, 55% dispersion in oil) and stirring at rt for 30 minutes (min). The mesylate/CD₃O⁻Na⁺ solution was stirred at rt for 6 h. Water (25 mL) was added to the reaction mixture and the solution was extracted with methyl t-butyl ether (2×15 mL). The organic layer was dried over Na₂SO₄ and concentrated in vacuo to give the product 38-d₅ (4.00 g, 85%).

Synthesis of 2-(Methoxy-d₃)-2,2-d₂-ethyl Methanesulfonate (16-d₅). To a solution of (2-(methoxy-d₃)-2,2-d₂-ethoxy)methyl)benzene 38-d₅ (4.00 g, 23.4 mmol) in THF (20 mL) was added 10% Pd/C (1.00 g) and the solution was subjected to hydrogenation for 6.0 h. The reaction mixture was then filtered through a pad of Celite. To the resulting filtrate was added triethylamine (5.22 mL, 37.5 mmol) and methanesulfonyl chloride (3.25 mL, 37.5 mmol) and the solution was stirred at rt overnight. To the resulting solution was added water (10 mL) and the mixture was extracted with EtOAc (2×10 mL). The organic layer was dried over Na₂SO₄ and evaporated to give the product 16-d₅ (2.50 g, 68%). ¹H NMR (400 MHz, CDCl₃): δ 4.40 (s, 2H), 3.10 (s, 3H).

II. Methoxy-2,2-d₂-ethyl Methanesulfonate (16-d₂). Intermediate 16-d₂ was synthesized as outlined in Scheme 3B above.

Synthesis of (2-Methoxy-2,2-d₂-ethoxy)methyl)benzene (38-d₂). To a solution of 2-benzyloxy-2,2-d₂-ethanol 37-d₂ (4.50 g, 28.9 mmol, Bird, I et al., see reference in Example 1BI above) in methylene chloride (40.0 mL) was added methanesulfonyl chloride (3.60 mL, 43.49 mmol) and triethylamine (6.00 mL, 43.5 mmol). The solution was stirred at rt for 1 h. Water (20 mL) was added to the reaction mixture and the organic layer was separated, washed with satd. brine solution (15 mL), dried over Na₂SO₄ and concentrated in vacuo. The resulting crude mesylate intermediate (6.00 g, 25.5 mmol, 80%) was dissolved in DMF (20.0 mL) and combined with CH₃O⁻Na⁺39, which was produced in situ by incubating CH₃OD [isotopic purity 99.8%, Aldrich] (2.00 mL, 55.9 mmol) in DMF (30.0 mL) with sodium hydride (2.20 g, 92.3 mmol, 60% dispersion in oil) and stirring at rt for 30 min. The mesylate/CD₃O⁻Na⁺ solution was stirred at rt for 6 h. Water (25 mL) was added to the reaction mixture and the solution was extracted with methyl t-butyl ether (2×15 mL). The organic layer was dried over Na₂SO₄ and concentrated in vacuo to give the product 38-d₂ (4.00 g, 85%).

Synthesis of Methoxy-2,2-d₂-ethyl Methanesulfonate (16-d₂). To a solution of (2-methoxy-2,2-d₂-ethoxy)methyl)benzene 38-d₂ (4.00 g, 23.08 mmol) in THF (70 mL) was added 10% Pd/C (1.00 g) and the solution was subjected to hydrogenation for 6.0 h. The resulting mixture was filtered through a pad of Celite. To the filtrate was added triethylamine (5.00 mL, 35.7 mmol) and methanesulfonyl chloride (2.30 mL, 35.7 mmol) and the solution was stirred at rt overnight. To the resulting solution was added water (10 mL) and the reaction mixture was extracted with EtOAc (2×10 mL). The organic layer was dried over Na₂SO₄ and concentrated in vacuo to give the product 16-d₂ (2.50 g, 68%). ¹H NMR (400 MHz, CDCl₃): δ 4.40 (s, 2H), 3.70 (s, 2H), 3.10 (s, 3H).

III. (Methoxy-d₃)ethyl Methanesulfonate (16-d₃). Intermediate 16-d₃ was synthesized as outlined in Scheme 3B, above.

Synthesis of 2-(Methoxy-d₃)ethyl Methanesulfonate (38-d₃). To a solution of 2-benzyloxyethanol 37 (8.10 g, 53.2 mmol, commercially available) in methylene chloride (80.0 mL) was added methanesulfonyl chloride 36 (6.60 mL, 79.7 mmol) and triethylamine (11.1 mL, 79.78 mmol). The reaction mixture was then stirred at rt for 1 h. Water (25 mL) was added and the organic layer was separated, washed with satd. brine solution (15 mL), dried over Na₂SO₄ and concentrated in vacuo. The resulting crude mesylate intermediate (3.73 mL, 92.0 mmol) was dissolved in DMF (100.0 mL) and combined with CD₃O⁻Na⁺39, which was produced in situ by incubating CH₃OD [isotopic purity 99.8%, Aldrich] (3.73 mL, 92.0 mmol) in DMF (100.0 mL) with sodium hydride (3.70 g, 151.8 mmol, 55% dispersion in oil) and stiffing at rt for 30 min. The mesylate/CD₃O⁻Na⁺ solution was stirred at rt for 6 h. Water (25 mL) was added to the mixture and the solution was extracted with methyl t-butyl ether (2×15 mL). The organic layer was dried over Na₂SO₄ and concentrated in vacuo to give the product 38-d₃ (7.00 g, 95%).

Synthesis of (Methoxy-d₃)ethyl Methanesulfonate (16-d₃). To a solution of (2-(methoxy-d₃)ethoxy)methyl)benzene 38-d₃ (2.70 g, 15.9 mmol) in THF (20 mL) was added 10% Pd/C (1.00 g, 50% wet). The mixture was subjected to hydrogenation for 6.0 h, and then was filtered through a pad of Celite. To the filtrate was added triethylamine (2.90 mL, 21.1 mmol) and methanesulfonyl chloride (1.70 mL, 21.1 mmol) and the solution was stirred at rt overnight. To the resulting solution was added water (10 mL) and the reaction mixture was extracted with EtOAc (2×10 mL). The organic layer was dried over Na₂SO₄ and concentrated in vacuo to give the product 16-d₃. (1.70 g, 77%). MS (M+H): 158.

### Example 2

### N-(3-Ethynylphenyl)-6,7-bis(2-(methoxy-d₃)ethoxy)quinazolin-4-amine hydrochloride (Compound 105)

Compound 105 was prepared as outlined in Scheme 1 above.

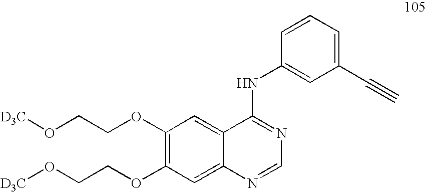

Synthesis of N-(3-Ethynylphenyl)-6,7-bis(2-(methoxy-d₃)ethoxy)quinazolin-4-amine hydrochloride (Compound 105). A solution of N-(3-ethynylphenyl)-6,7-dihydroxy-4-quinazolinamine 15 (500 mg, 1.80 mmol, prepared as described by Ramanadhan, J P et al. in WO 2007060691A2), 2-(methoxy-d₃)ethyl methanesulfonate 16-d₃ (1.10 g, 7.21 mmol), and cesium carbonate (2.34 g, 7.21 mmol) in CH₃CN (10 mL) was heated to 50° C. and stirred for 2 h. The reaction mixture was cooled to rt and concentrated in vacuo. The residue was dissolved in cold water and the solution was extracted with ethyl acetate (2×10 mL). The combined organic layers were washed with water and brine, dried over Na₂SO₄ and concentrated in vacuo. The crude compound was subjected to column:chromatography (3:7 acetone:hexane) to yield Compound 105 (300 mg; 45%)). ¹H NMR (400 MHz, DMSO-d₆): δ 11.1 (s, 1H), 8.90 (s, 1H), 8.20 (s, 1H), 7.90 (s, 1H), 7.70-7.80 (m, 1H), 7.40-7.50 (m, 1H), 7.30-7.40 (m, 1H), 7.20 (s, 1H), 4.40 (m, 4H), 4.30 (s, 1H), 3.80 (m, 4H). MS (M+H): 400 (M⁺+1).

### Example 3

### Synthesis of N-(3-(Ethynyl-d₁)phenyl)-6,7-bis(2-methoxyethoxy) quinazolin-4-amine hydrochloride (Compound 110)

Compound 110 was synthesized as outlined in Scheme 2 above

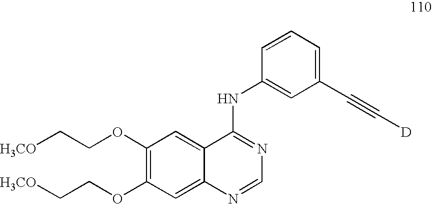

Synthesis of N-(3-(Ethynyl-d₁)phenyl)-6,7-bis(2-methoxyethoxy)quinazolin-4-amine (Compound 110). To a solution of erlotinib (250 mg, 0.90 mmol, prepared as described in Knesl, P et al., Molecules, 2006, 11:286-297) in THF (20 L) cooled to −30° C. was added isopropyl magnesium chloride (1.6 N in THF, 2 mL). The reaction mixture was stirred at −30° C. for 3.0 h, CD₃OD (1 mL) was added and the mixture was slowly brought to rt over a period of 2.0 h then was stirred at rt. After 10.0 h the reaction mixture was quenched with D₂O and extracted with ethyl acetate (2×25 mL). The combined organic extracts were washed with water and brine, dried over Na₂SO₄ and concentrated in vacuo to give Compound 110 (178 mg, 76%).

### Example 4

### Synthesis of N-(3-ethynylphenyl)-6,7-bis(2-methoxyethoxy)quinazolin-4-amine (Compound 106)

Compound 106 was prepared as outlined in Scheme 1, above.

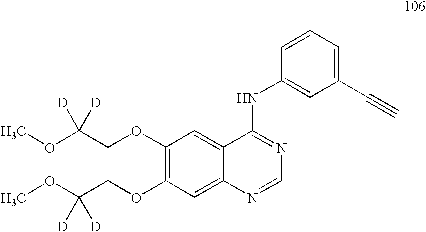

Synthesis of N-(3-Ethynylphenyl)-6,7-bis(2-methoxyethoxy)quinazolin-4-amine (Compound 106). To a solution of the dihydroxy compound 15 (0.600 g, 2.16 mmol, see Example 2) in DMF (15 mL) was added K₂CO₃ (1.20 g, 8.64 mmol) and 2-methoxy-2,2-d₂-ethyl methanesulfonate 16-d₂ (0.744 g, 4.76 mmol). The solution was stirred at rt for 15 min and then heated to 60° C. for 2 h. DMF was removed in vacuo and water (10 mL) was added to the residue. The resulting solution was extracted with EtOAc (2×5 mL) and the organic layer was dried over Na₂SO₄ and concentrated in vacuo. The product was taken up in diethyl ether (3 mL) and the solids were filtered to give the product, Compound 106 (0.280 g, 54%). ¹H NMR (400 MHz, DMSO-d₆): δ 2.30 (s, 3H), 2.40 (s, 3H), 4.10 (s, 1H), 4.20 (s, 4H), 7.10 (m, 1H), 7.30 (m, 1H), 7.80 (m, 1H), 7.90 (s, 1H), 8.40 (s, 1H), 9.40 (s, 1H). MS (M+H): 398.

### Example 5

### Synthesis of N-(3-(Ethynyl-d₁)phenyl)-6,7-bis(2-(methoxy-d₃)-2,2-d₂-ethoxy) quinazolin-4-amine (Compound 109)

Compound 109 was prepared as outlined in Scheme 1, above.

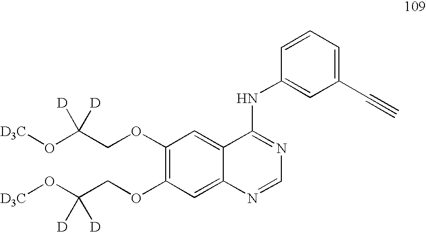

Synthesis of N-(3-(Ethynyl-d₁)phenyl)-6,7-bis(2-(methoxy-d₃)-2,2-d₂-ethoxy) quinazolin-4-amine (Compound 109). To a solution of the dihydroxy compound 15 (0.500 g, 1.80 mmol, see Example 2) in CH₃CN (8.0 mL) was added Cs₂CO₃ (2.35 g, 7.21 mmol) and 2-(methoxy-d₃)-2,2-d₂-ethyl methanesulfonate 16-d₅ (1.43 g, 9.01 mmol). The solution was stirred at rt for 15 min, then heated to reflux for 6 h. CH₃CN was removed in vacuo and to the residue was added water (10 mL). The solution was extracted with EtOAc (2×5 mL) and the EtOAc layer was dried over Na₂SO₄ and concentrated in vacuo. The crude product was subjected to column chromatography with hexane:acetone (8:2). The resulting product was added to HCl in diethyl ether (3 mL) and the solids were filtered to give Compound 109 (0.100 g, 43%). ¹H NMR (400 MHz, DMSO-d₆): δ 4.20 (s, 1H), 4.30 (s, 4H), 7.30 (m, 1H), 7.40 (m, 1H), 7.50 (m, 1H), 7.80 (m, 1H), 7.90 (s, 1H), 8.20 (s, 1H), 8.90 (s, 1H), 11.0 (s, 1H). MS (M+H): 404.

### Example 6

### Metabolism Studies in Human Liver Microsomes

The metabolic stability of compounds of the invention was tested using pooled liver microsomal incubations. Full scan LC-MS analysis was then performed to detect major metabolites. Samples of the test compounds, exposed to pooled human liver microsomes, were analyzed using HPLC-MS (or MS/MS) detection. For determining metabolic stability, multiple reaction monitoring (MRM) was used to measure the disappearance of the test compounds. Q1 full scans were used to detect the major metabolites.

Experimental Procedures. Human liver microsomes (“HLM”; 20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Stock solutions of test compounds (7.5 mM) were prepared in DMSO. The 7.5 mM stock solutions were diluted to 50 μM in acetonitrile (ACN). The 20 mg/mL human liver microsomes were diluted to 0.625 mg/mL in 0.1 M potassium phosphate buffer, pH 7.4, containing 3 mM MgCl₂. The diluted microsomes (375 μL) were added to wells of a 96-well deep-well polypropylene plate in triplicate. Ten μL of the 50 μM test compound solution was added to the microsomes and the mixture was pre-warmed for 10 minutes. Reactions were initiated by addition of 125 μL of pre-warmed NADPH solution (8 mM NADPH in 0.1M potassium phosphate buffer, pH 7.4, containing 3 mM MgCl₂). The final reaction volume was 0.5 mL and contained 0.5 mg/mL human liver microsomes, 1 μM test compound, and 2 mM NADPH in 0.1 M potassium phosphate buffer, pH 7.4, and 3 mM MgCl₂. The reaction mixtures were incubated at 37° C., and 50 μL aliquots were removed at 0, 5, 10, 20, and 30 minutes and added to shallow-well 96-well plates which contained 50 μL of ice-cold ACN with internal standard to stop the reactions. The plates were stored at 4° C. for 20 minutes after which 100 μL of water was added to the wells of the plate before centrifugation to pellet precipitated proteins. Supernatants were transferred to another 96-well plate and analyzed for amounts of parent remaining by LC-MS/MS using an Applied Bio-systems API 4000 mass spectrometer.

The in vitro t_{1/2}s for test compounds were calculated from the slopes of the linear regression of % parent remaining (ln) vs incubation time relationship. in vitro t_{1/2}=0.693/k, where k=−[slope of linear regression of % parent remaining(ln) vs incubation time]. Data analysis was performed using Microsoft Excel Software. The results of these experiments are depicted in Table 3:

TABLE 3
Stability of Compounds in Human Liver Microsomes.

| Compound | t_{1/2} (min) |
|---|---|
| erlotinib | 57.8 |
| 109 | 83.5 |
| 110 | 42.7 |
| 106 | 43.8 |
| 105 | 62.3 |

Under the assay conditions tested (0.5 mg/mL HLM, 1 mM test compound) Compound 109 demonstrated 44% longer t_{1/2} compared to erlotinib.

### Example 7

### Pharmacokinetic Studies in Mice

Three male BALB/c nude mice (nu/nu) were each administered a combination of 5 mg/kg of Compound 109 and 5 mg/kg of erlotinib in PBS by oral gavage. Blood samples were collected at 5, 15, 30, 60, 180, 360, and 1440 min from each animal via saphenous vein, with 20-30 μL being withdrawn at each time point. Fifteen microliters of blood sample were then submitted for quantitative bioanalysis by HPLC-MS/MS. Fundamental pharmacokinetic parameters were obtained from the non-compartmental analysis using WinNonlin. The results of this experiment are shown in Table 4.

TABLE 4
Pharmacokinetic Properties of Compounds
After Oral Dosing in Mice.

| Compound | Cmax (ng/mL) | AUC (hr * ng/mL) |
|---|---|---|
| 109 | 1288 | 2126 |
| erlotinib | 953 | 1485 |
| % change | +35 | +43 |

Under the conditions tested, Compound 109 demonstrated a 35% higher Cmax and a 43% higher AUC as compared to erlotinib.

### Example 8

### Proliferation Studies in EFG-Stimulated A-431 Cells

The compounds of the invention were tested for their ability to inhibit proliferation of human A-431 cells in vitro using the procedure of Handler, J A et al, J Biol Chem 1990, 265:3669. Human A-431 cells were stimulated with 1 ng/ml of EGF and grown in the presence of ³H-thymidine (0.05 μCi) and varying concentrations (5 nM-10 μM) of test compound (Compound 105, Compound 109, Compound 110, erlotinib and staurosporine as a control) for 24 h at 37° C. ³H-thymidine incorporation was measured by scintillation counting.

The results for each concentration of compound tested were expressed as a percent of control specific activity ((measured specific activity/control specific activity)×100) obtained in the presence of the test compounds.

The IC₅₀ values (concentration causing a half-maximal inhibition of control specific activity) and Hill coefficients (nH) were determined by non-linear regression analysis of the inhibition curves generated with mean replicate values using Hill equation curve fitting:

(Y=D+[(A−D)/(1+(C/C₅₀)'^{nH})],

where Y=specific activity, D=minimum specific activity, A=maximum specific activity, C=compound concentration, C₅₀=IC₅₀ and nH=slope factor). This analysis was performed using a software developed at Cerep (Hill software) and validated by comparison with data generated by the commercial software SigmaPlot® 4.0 for Windows® (© 1997 by SPSS Inc.). The results are shown below in Table 5.

TABLE 5
Anti-proliferative Activity in A-431 Cells.

| Compound | IC₅₀ (μM) |
|---|---|
| Erlotinib | 0.28 |
| 105 | 0.23 |
| 109 | 0.24 |
| 110 | 0.11 |

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

We claim:

1. A compound of any one of the formulae: 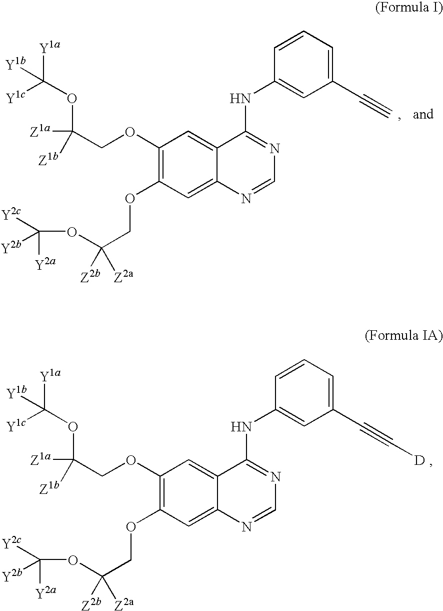
or a pharmaceutically acceptable salt of either of said compounds, wherein:
each Y is deuterium; and
each Z is deuterium.

2. The compound of claim 1, wherein any atom not designated as deuterium in any of the embodiments set forth above is present at its natural isotopic abundance.

3. A pyrogen-free pharmaceutical composition comprising a compound of claim 1, and a pharmaceutically acceptable carrier.

4. The composition of claim 3, further comprising a second therapeutic agent useful in the treatment of a disease or disorder selected from cancer, inflammation, angiogenesis, vascular restenosis, immunological disorder, pancreatitis, kidney disease, blastocyte maturation and implantation, psoriasis, and benign prostatic hypertrophy (BPH).

5. The composition of claim 4, wherein the second therapeutic agent is selected from 2-deoxy-2-[¹⁸F]fluoro-D-glucose, 3′-deoxy-3′-[¹⁸F]fluorothymidine, 5-fluorouracil, AV412, avastin, bevacizumab, bexarotene, bortezomib, calcitriol, canertinib, capecitabine, carboplatin, celecoxib, cetuximab, CHR-2797, cisplatin, dasatinib, digoxin, enzastaurin, etoposide, everolimus, fulvestrant, gefitinib, gemcitabine, genistein, imatinib, irinotecan, lapatinib, lenalidomide, letrozole, leucovorin, matuzumab, oxaliplatin, paclitaxel, panitumumab, pegfilgrastim, pegylated alfa-interferon, pemetrexed, Polyphenon® E, satraplatin, sirolimus, sorafenib, sutent, sulindac, sunitinib, taxotere, temodar, temozolomide, temsirolimus, TG01, tipifarnib, trastuzumab, valproic acid, vinflunine, volociximab, vorinostat, and XL647.

6. The composition of claim 5, wherein the second therapeutic agent is bevacizumab.

7. A method of treating a patient suffering from or susceptible to a disease or disorder selected from cancer, inflammation, angiogenesis, vascular restenosis, immunological disorder, pancreatitis, kidney disease, blastocyte maturation and implantation, psoriasis, and benign prostatic hypertrophy (BPH), comprising the step of administering to the patient in need thereof a composition of claim 3.

8. The method of claim 7, wherein the patient is suffering from or susceptible to a cancer selected from non-small cell lung cancer, ovarian cancer, colorectal cancer, head and neck cancer, brain cancer, bladder cancer, sarcoma, prostate cancer, melanoma, cervical cancer, solid tumors, astrocytoma, breast cancer, pancreatic cancer, glioblastoma multiform, renal cancer, digestive/gastrointestinal cancer, liver cancer, and gastric cancer.

9. The method of claim 8, wherein the patient is suffering from non-small cell lung cancer.

10. The method of claim 7, comprising the further step of co-administering to the patient in need thereof a second therapeutic agent useful in the treatment of a disease or disorder selected from cancer, inflammation, angiogenesis, vascular restenosis, immunological disorder, pancreatitis, kidney disease, blastocyte maturation and implantation, psoriasis, and benign prostatic hypertrophy (BPH).

11. The method of claim 10, wherein the patient is suffering from cancer and the second therapeutic agent is selected from 2-deoxy-2-[¹⁸F]fluoro-D-glucose, 3′-deoxy-3′-[¹⁸F]fluorothymidine, 5-fluorouracil, AV412, avastin, bevacizumab, bexarotene, bortezomib, calcitriol, canertinib, capecitabine, carboplatin, celecoxib, cetuximab, CHR-2797, cisplatin, dasatinib, digoxin, enzastaurin, etoposide, everolimus, fulvestrant, gefitinib, gemcitabine, genistein, imatinib, irinotecan, lapatinib, lenalidomide, letrozole, leucovorin, matuzumab, oxaliplatin, paclitaxel, panitumumab, pegfilgrastim, pegylated alfa-interferon, pemetrexed, Polyphenon® E, satraplatin, sirolimus, sorafenib, sutent, sulindac, sunitinib, taxotere, temodar, temozolomide, temsirolimus, TG01, tipifarnib, trastuzumab, valproic acid, vinflunine, volociximab, vorinostat, and XL647.

12. The method of claim 11, wherein the second therapeutic agent is bevacizumab.

13. The method of claim 12, wherein the patient is suffering from non-small cell lung cancer.
